(19) United States
(12) Patent Application Publication
(10) Pub. No.: US 2017/0198245 A1
(43) Pub. Date: Jul. 13, 2017

(54) METHOD FOR MANUFACTURING MICRO-HEMISPHERE ARRAY PLATE, MICROFLUIDIC DEVICE COMPRISING MICRO-HEMISPHERE ARRAY PLATE, AND METHOD FOR CULTURING CELL AGGREGATE USING SAME

(71) Applicant: MICROFIT CO., LTD., Seoul (KR)

(72) Inventors: Hyun-Jik OH, Hanam-si (KR); Da-Yoon NO, Gimpo-si (KR); Sang-Hoon LEE, Ansan-si (KR)

(73) Assignee: MICROFIT CO., LTD.

(21) Appl. No.: 15/472,290

(22) Filed: Mar. 29, 2017

Related U.S. Application Data
(63) Continuation of application No. PCT/KR2014/009070, filed on Sep. 29, 2014.

(51) Int. Cl. C12M 3/06; C12M 3/00; B29C 45/26; C12N 5/00; B29C 45/00; B05D 1/00

(52) U.S. Cl. CPC C12M 23/16; B29C 45/0001; B05D 1/005; B29C 45/26; C12N 5/0062; C12M 21/08; B29L 2031/756

(57) ABSTRACT

The present invention relates to a method for manufacturing a micro-hemisphere array plate, a microfluidic device comprising the micro-hemisphere array plate, and a method for culturing a cell aggregate using the same. It is possible to form a cell aggregate having excellent condition if cells are cultured in three dimensions by using the method for manufacturing a micro-hemisphere array plate, the microfluidic device comprising the micro-hemisphere array plate, and the method for culturing a cell aggregate using the same according to the present invention. In particular, because cells are cultured in a condition more similar to an environment within the human body, it is possible to form a cell aggregate having a better condition than by existing cell-culturing methods.

### CROSS-REFERENCES TO RELATED APPLICATIONS

This application claims the benefit of priority and is a Continuation application of the prior International Patent Application No. PCT/KR2014/009070, with an international filing date of Sep. 29, 2014, which designated the United States, the entire disclosures of all applications are expressly incorporated by reference in their entirety herein.

### BACKGROUND OF THE INVENTION

1. Field of the Invention

The present invention relates to a method of manufacturing a micro-hemisphere array plate, a microfluidic device including a micro-hemisphere array plate manufactured thereby, and a method of culturing a cell aggregate using the same.

2. Description of Related Art

Cells in the human body form a three-dimensionally shaped aggregate through interaction between neighboring cells and the extracellular matrix. Such a three-dimensional shape plays a very important role, biochemically and mechanically, in cell physiology. In particular, cell aggregation formed as a three-dimensional shape plays a very important role in studies on cells constituting general tissues or organs, cancer cells, and stem cells, for clinical development of new drugs, or for differentiation using stem cells.

However, in general, it is very difficult to culture cells to have a three-dimensional shape. In particular, it is more difficult to three-dimensionally culture human primary cells. Due to these problems, cells are generally cultured two-dimensionally and thus used in drug screening or various experiments. However, two-dimensional culturing is performed in very different environments from actual in vivo environments, and thus intrinsic characteristics or tissue specificity of cells to be used in experiments are lost and, consequently, it is very difficult to obtain desired experimental results.

Thus, three-dimensional culturing of cells in vitro is very important and various studies thereon are ongoing. Such three-dimensional culture may be performed, for example, by hanging-drop culture or a non-adhesive surface method, or using a spinner flask, a rotary system, or the like. However, these culture methods are not simple, it is difficult to achieve mass-production thereby, and it is difficult to appropriately adjust the shape, size or number of cells.

To address these problems, Korean Patent Application Publication No. 10-2013-0013537 (Patent Document 1) as prior art discloses a method of manufacturing a hemispherical micro-well using surface tension and formation of a cell aggregate using the same. However, the method disclosed in Patent Document 1 is not a method of precisely manufacturing a micro-hemisphere, and thus it is impossible to form a complete hemisphere and, accordingly, even though a cell aggregate is formed, the cell aggregate cannot be completely separated from a micro-well in a collecting process, cells are separated to the outside of a micro-well even by slight impact, and the shapes of the collected cells and cell aggregate are destroyed. In addition, cells are cultured in a state in which environments similar to fluid flow in the human body are not formed, and thus the state of a formed cell aggregate is not good.

### BRIEF SUMMARY OF THE INVENTION

Therefore, the present invention has been made in view of the above problems, and it is one object of the present invention to provide a micro-hemisphere array plate capable of forming a cell aggregate in better condition and a microfluidic device including a micro-hemisphere array plate. In particular, when a microfluidic device including a micro-hemisphere array plate is used, a certain amount of fluid flows and thus an environment similar to that in the human body is formed, and cells are cultured therein to thereby form a better cell aggregate and also to form an aggregate of primary cells, which are generally difficult to form as an aggregate of cells.

The above and other objects can be accomplished by the present invention described below.

In accordance with one aspect of the present invention, provided is a method of manufacturing a micro-hemisphere array plate, including: 1) attaching a photosensitive photoresist to a silicon substrate; 2) adjusting a height of the photosensitive photoresist by spin coating; 3) hemispherically etching the photoresist by overcuring; 4) depositing a primary metal layer on an etched surface of the photoresist after step 3); 5) depositing a secondary metal layer on the primary metal layer after the depositing; 6) forming a mold core layer on the secondary metal layer; 7) planarizing an upper surface of the mold core layer after step 6); 8) separating the mold core layer after step 7); 9) injection-molding a micro-hemisphere array plate by using the separated mold core layer as a mold; and 10) imparting hydrophilicity or hydrophobicity to a surface of the micro-hemisphere array plate.

In accordance with another aspect of the present invention, provided is a microfluidic device including a micro-hemisphere array plate, including: a sample inlet through which a sample including a single cell or a plurality of cells and a cell culture is injected via a single or plurality of channels; a sample mixing part connected to the sample inlet, allowing the sample to be mixed therein while moving, wherein the moving is performed via a single or plurality of channels, the single or plurality of channels having a zigzag form, the single or plurality of channels being repetitively disposed in a pyramid form through a plurality of steps, and the steps being configured such that a lower step further comprises one or more channels than in an upper step, and including a flow channel connecting the steps to one another; and a cell aggregate formation part connected to the sample mixing part, connected to channels constituting the lowermost step among the steps, and including a plurality of micro-hemisphere array plates in which the single or mixed cells in the mixed sample are three-dimensionally cultured to form a cell aggregate.

As is apparent from the fore-going description, when cells are three-dimensionally cultured using a method of manufacturing a micro-hemisphere array plate, a microfluidic device including a micro-hemisphere array plate manufactured thereby, and a method of culturing a cell aggregate using the same, it is possible to form a cell aggregate having excellent condition. In particular, since cells are cultured under conditions more similar to an environment in the human body, a cell aggregate having better condition may be formed as compared to existing cell culturing methods. The cell aggregate cultured through the present invention may be directly used in cell treatment, and cells that are difficult to obtain artificially may be obtained by culturing. In addition, since cells are cultured under conditions similar to the human body, it is possible to form a cell aggregate of cells that are difficult to agglomerate three-dimensionally. In addition, two or more types of cells may be co-cultured under conditions similar to the human body, and thus a high-quality cell aggregate may be formed. Consequently, the present invention may contribute to achieving innovative development in drug screening or cytotoxicity, and in a variety of tests.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates a process of manufacturing a micro-hemisphere array plate according to Example 1;

FIGS. 2A and 2B illustrate images of the micro-hemisphere array plate manufactured according to Example 1;

FIG. 3 illustrates a process of culturing hADSCs using the micro-hemisphere array plate of Example 1;

FIG. 4 illustrates three-dimensional co-culturing using the micro-hemisphere array plate of Example 1;

FIG. 5 is a cross-sectional view of a microfluidic device including a micro-hemisphere array plate according to Example 2;

FIG. 6 is an image of the microfluidic device including a micro-hemisphere array plate according to Example 2;

FIGS. 7A-7C illustrate images showing three-dimensional culturing of human cells that form a cell aggregate, using the micro-hemisphere array plate of Example 1;

FIGS. 8A-8B illustrate images showing formation of a cell aggregate in the cases of Comparative Example 1 and Example 1;

FIGS. 9A-9E illustrate images showing comprehensive comparison between the cases of Comparative Example 2 and Example 1;

FIGS. 10A-10B illustrate images showing formation of a cell aggregate using human hepatocytes with or without fluid flow as in Example 2;

FIGS. 11A-11B illustrates images showing results of culturing human primary hepatocytes with and without fluid flow as in Example 2;

FIGS. 12A-12F illustrate images comprehensively comparing the case of Comparative Example 2 with the case of Example 2;

FIGS. 13A-13D illustrate images showing results of three-dimensionally co-culturing human liver cells and hADSCs using the micro-hemisphere array plate of Example 1;

FIGS. 14A-14E illustrate graphs and images showing functional measurement test results when human liver cells and hADSCs are three-dimensionally co-cultured using the micro-hemisphere array plate of Example 1;

FIGS. 15A-15F illustrate TEM images of the inside of a three-dimensionally co-cultured cell aggregate using the micro-hemisphere array plate of Example 1;

FIGS. 16A-16B illustrate images showing excellent cell aggregate three-dimensionally co-cultured using the micro-hemisphere array plate of Example 1 when removed from a hemisphere;

FIGS. 17A-17C illustrate images showing results of two-dimensionally co-culturing human primary hepatocytes and hADSCs;

FIGS. 18A-18B illustrate images showing staining results of two-dimensionally co-cultured human primary hepatocytes and hADSCs;

FIGS. 19A-19B illustrate images showing active albumin secretion when human primary hepatocytes and hADSCs were co-cultured on 3D in Example 1; and

FIGS. 20A-20F illustrate images comprehensively comparing the cases in which human primary hepatocytes and hADSCs were co-cultured on 2D and 3D.

### DETAILED DESCRIPTION OF THE INVENTION

Therefore, the inventors of the present invention conducted intensive research to develop a method of manufacturing a micro-hemisphere array plate, whereby a cell aggregate in better condition may be formed and culturing conditions may be provided in an environment similar to the human body, and a microfluidic device including a micro-hemisphere array plate manufactured thereby, and, as a result, they discovered a method of manufacturing a micro-hemisphere array plate, according to the present invention, a microfluidic device including a micro-hemisphere array plate manufactured thereby, and a method of culturing a cell aggregate using the same, thus completing the present invention.

In particular, a method of manufacturing a micro-hemisphere array plate, according to the present invention, includes: 1) attaching a photosensitive photoresist to a silicon substrate; 2) adjusting the height of the photosensitive photoresist by spin coating; 3) hemispherically etching the photoresist through overcuring; 4) depositing a primary metal layer on the etched surface after step 3); 5) depositing a secondary metal layer on the primary metal layer; 6) forming a mold core layer on the secondary metal layer; 7) planarizing an upper surface of the mold core layer after step 6); 8) separating the mold core layer therefrom after step 7); 9) injection-molding a micro-hemisphere array plate using the separated mold core layer as a mold; and 10) imparting hydrophilicity or hydrophobicity to a surface of the micro-hemisphere array plate.

In step 1), the silicon substrate is suitable for use in attaching the photosensitive photoresist.

Photosensitive photoresists are divided into negative photoresists in which, when a photosensitive photoresist is exposed, generally, to ultraviolet light (UV, 350 nm to 400 nm), a region in which the light is received is cross-linked and thus remains, and positive photoresists in which a region in which light is received is developed. The photosensitive photoresist is suitable for use in forming a hemisphere after etching, and may include both negative and positive photoresists.

The length of the photosensitive photoresist to be attached in step 1) may range from 100 μm to 1,000 μm. When the length of the photosensitive photoresist is less than 100 μm, the diameter and depth thereof are too small and thus it is difficult to form a hemisphere after etching. When the length of the photosensitive photoresist is greater than 1,000 μm, the size of a hemisphere after etching is too large and thus it is difficult for cultured cells to form an aggregate and it is difficult to maximize intrinsic characteristics of cells. In addition, when the length of the photosensitive photoresist is within the above-described range, a cell aggregate in excellent condition may be formed under optimum conditions. In addition, within the above-described length range, the depth of a hemisphere may be adjusted by adjusting a coating height and temperature conditions of the photosensitive photoresist.

In addition, when spin coating is performed as in step 2), the height of the photosensitive photoresist may be adjusted.

As in step 3), the photosensitive photoresist may be etched through overcuring to form a hemisphere. Overcuring is a process wherein, when the photosensitive photoresist is heated at a suitable temperature or higher, an edge portion turns into a round form having a curved shape. As such, when overcuring is performed, a hemisphere may be more precisely formed than when using an existing method of manufacturing a hemispherical micro-well, and thus a cell aggregate in excellent condition may be formed, and, even after culturing, the cell aggregate may be separated from a micro-hemisphere array plate without damage to the state of the aggregate.

In step 4), the primary metal layer is deposited on the etched surface of the photosensitive photoresist. The deposition method is not particularly limited and is preferably chemical vapor deposition, physical vapor deposition, or the like. The deposited primary metal layer is used to more easily separate the mold core layer and may be formed of at least one selected from the group consisting of Cr, Ti, Au, Ni, Cu, Al, and Fe. A deposition height of the primary metal layer may range from 100 μm to 500 μm. When the height of the primary metal layer is less than 100 μm, adhesion of the secondary metal layer to a thin film is weakened and thus it is not suitable to form the secondary metal layer to a large height. When the height of the primary metal layer is greater than 500 μm, a peeling phenomenon, in which a seed metal layer peels off, occurs.

As in step 5), after deposition of the primary metal layer, the secondary metal layer may be deposited thereon. The secondary metal layer is used to facilitate electroplating of a mold core and may be formed of the same material as the mold core or at least one selected from the group consisting of Au, Ag, Pt, Ni, and Cu, which have high electrical conductivity. The reason for separately depositing the primary and secondary metal layers is that, when the secondary metal layer is used alone, thin film adhesion deteriorates and thus it is difficult to form a mold core to a desired height during electroplating. The deposition method of the secondary metal layer is not particularly limited, and is preferably chemical vapor deposition, physical vapor deposition, or the like. A deposition height of the secondary metal layer may range from 1,000 μm to 2,000 μm. When the deposition height of the secondary metal layer is less than 1,000 μm, stress of a thin film itself is weak and thus plating is not suitable. When the deposition height of the secondary metal layer is greater than 2,000 μm, surface roughness (RMS) increases and thus it may affect uniformity of the mold core layer in step 6).

In step 6), the mold core layer is formed on the secondary metal layer. In this regard, the mold core layer may be formed by, preferably, electroplating, which is suitable for use in forming a metal layer to a large height. The mold core layer may be formed of at least one selected from the group consisting of nickel, titanium, and aluminum, which have sufficient strength for use as a mold core.

In step 7), the upper surface of the mold core layer formed in step 6) is planarized. By planarizing the mold core layer, the micro-hemisphere array plate injection-molded in step 9) may be smoothly mounted in a mold, and injection moldability of the micro-hemisphere array plate to be manufactured may be enhanced. The planarization method may be used without limitation so long as it is a method that can planarize the mold core layer to achieve leveling of the micro-hemisphere array plate, and is preferably chemical mechanical planarization (CMP), bright dipping, tumbling barreling, buffing, belt sanding, picking, or the like.

In step 8), the mold core layer is separated. The separation method of the mold core layer is not particularly limited and is preferably a separation method wherein the silicon substrate is removed by melting with KOH, TMAH, or the like and the remaining portion of the primary metal layer is removed using an etching solution. In addition, in this process, although not particularly limited, when the secondary metal layer differs from the mold core, the secondary metal layer may also be separated.

In step 9), the micro-hemisphere array plate is injection-molded using the mold core layer as a mold. The injection-molding method may be used without particular limitation so long as it is suitable for injection-molding of a micro-hemisphere array plate.

Any material used for injection-molding may be used without particular limitation so long as it can be injection-molded and may be at least one selected from the group consisting of polycarbonate (PC), polymethylmethacrylate (PMMA), polystyrene (PS), and cyclic olefin copolymer (COC).

In step 10), hydrophilicity or hydrophobicity may be imparted to the surface of the micro-hemisphere array plate. A method of imparting hydrophilicity or hydrophobicity is not particularly limited and is preferably a method of adjusting the degree of hydrophilicity or hydrophobicity of the surface thereof through a plasma method or chemical surface treatment. In addition, when a cell aggregate is cultured through the micro-hemisphere array plate finally manufactured by such surface modification, formation of air bubbles in a hemisphere may be minimized, thereby maximizing formation of a three-dimensional cell aggregate.

The hemisphere of the micro-hemisphere array plate manufactured using the above-described manufacturing method may have a diameter of 100 μm to 1,000 μm. When the diameter of the hemisphere is within the above-described range, a three-dimensional cell aggregate in better condition may be formed.

According to the method of manufacturing a micro-hemisphere array plate, according to the present invention, a hemisphere and a hemisphere array are formed to a more precise shape than in an existing method of manufacturing a hemispherical micro-well. Accordingly, a cell aggregate is formed more three-dimensionally.

In addition, when a cell aggregate is cultured using a micro-hemisphere array plate manufactured using the method of manufacturing a micro-hemisphere array plate, according to the present invention, the cell aggregate is more satisfactorily formed than when an existing method is used. In addition, the formed cell aggregate may be separated from the micro-hemisphere array plate without damage to the cell aggregate. This indicates that the state of the formed cell aggregate is far better than when an existing two-dimensional culturing method is used.

A microfluidic device including a micro-hemisphere array plate manufactured by the above-described manufacturing method, according to another aspect of the present invention, includes: a sample inlet 1 through which a sample including a single cell or a plurality of cells and a cell culture is injected via one or more channels 4; a sample mixing part 2 connected to the sample inlet 1, allowing the sample to be mixed therein while moving, in which the moving is performed via one or more channels, the one or more channels having a zigzag form, the one or more channels being repetitively disposed in a pyramid form through a plurality of steps, and the steps being configured such that a lower step further includes one or more channels than in an upper step, and including a flow channel 5 connecting the steps to one another; and a cell aggregate formation part 3 connected to the sample mixing part 2, connected to channels constituting the lowermost step among the steps, and including a plurality of micro-hemisphere array plates in which the single or mixed cells in the mixed sample are three-dimensionally cultured to form a cell aggregate.

The microfluidic device according to the present invention allows a single or plurality of cells to three-dimensionally form a cell aggregate, and the cell aggregate is formed under conditions similar to an environment in the human body by passing fluid therethrough. Adults contain an average of about 60% water as the most vital material constituting the human body, and thus cells in the human body form an aggregate in a state in which fluidic motion such as blood flow or the like occurs. Accordingly, the present invention provides conditions similar to an environment in the human body and thus a cell aggregate having better quality is formed.

The sample inlet may allow a cell and a cell culture to be injected therethrough. The cell may be a single cell or a plurality of cells, and the injected cells forms a cell aggregate in the micro-hemisphere array plate. The sample including a single cell or a plurality of cells and a cell culture may be injected via the sample inlet at a flow rate of 10 nl/min to 10 μl/min, and injection of the sample at a flow rate within the above-described range is similar to an environment in the human body. In particular, when the flow rate is less than 10 nl/min, it is largely outside conditions similar to the human body and it is also difficult to remove unnecessary cells around the hemisphere. When the flow rate is greater than 10 μl/min, it is difficult for cells to precipitate in the hemisphere of the micro-hemisphere array plate. The cell culture injected through the sample inlet may be used without particular limitation so long as it can culture cells and flows as a fluid. In addition, an entrance of the sample inlet through which the sample is injected may be a single or plurality of channels, and the sample may be injected via a single or plurality of paths.

Meanwhile, the sample mixing part allows the sample to be mixed therein while moving. In this regard, the sample may move via a single or plurality of channels so that the sample can be more easily mixed. The channel may have a diameter of 500 μm to 2.0 mm. When the diameter of the channel is less than 500 μm, the number of micro-hemisphere arrays decreases and a fluid pressure of the channel increases. When the diameter of the channel is greater than 2.0 mm, it is difficult to move the sample under conditions similar to the human body, and it is not easy to control micro-hemisphere arrays. In addition, to more vigorously mix the sample, the single or plurality of channels may be in a zigzag form. The single or plurality of channels in a zigzag form is repetitively arranged in a pyramid form through a plurality of steps. When the steps are repeated in a pyramid form, mixing of the sample may be actively performed and a concentration gradient according to chamber structure may be formed. In addition, to obtain the pyramid form, among the steps, a lower step may further include one or more channels than in an upper step. In addition, the steps are connected by flow channels connecting the steps to one another. The sample mixing part has an excellent effect of mixing the sample through the above-described configuration.

The cell aggregate formation part is connected to the sample mixing part and to the lowermost step among the steps. In addition, the single or mixed cells in the mixed sample are three-dimensionally cultured in a plurality of micro-hemisphere array plates to form a cell aggregate. The micro-hemisphere array plates may not decrease a flow rate of a culture and may provide an environment more similar to that in the human body as compared to a single micro-hemisphere array plate, thereby forming an excellent cell aggregate. In addition, although not particularly limited, the number of micro-hemisphere array plates is preferably the same as the number of channels included in the lowermost step among the steps. This is because the micro-hemisphere array plate is directly connected to each of the channels constituting the lowermost step and thus a high-quality cell aggregate may be formed in an environment more similar to the human body without inhibiting transfer of the sample up to the previous steps.

The micro-hemisphere array plate is not particularly limited, but the micro-hemisphere array plate manufactured using the method of manufacturing a micro-hemisphere array plate, according to another aspect of the present invention, may be used to form a higher-quality cell aggregate, whereby formation of the hemisphere is reproduced at a much higher rate than in an existing method. In addition, the formed cell aggregate may be separated from the micro-hemisphere array plate without damage thereto. This indicates that the state of the cell aggregate is far better than in an existing two-dimensional culturing method.

Meanwhile, the single or mixed cells precipitate in the hemisphere of the micro-hemisphere array plate to form a cell aggregate, and impurities and unnecessary cells present around the hemisphere of the micro-hemisphere array plate are removed by a flow rate of the remaining sample flowing above the hemisphere. By repeating these processes numerous times, a cell aggregate is formed and cultured in the hemisphere of the micro-hemisphere array plate.

The microfluidic device including a micro-hemisphere array plate, according to the present invention, has three main functions. First, the microfluidic device has a concentration gradient function that allows the sample including a cell, a cell culture, and the like to flow up to each cell aggregate formation part according to concentration. Second, the microfluidic device includes a functional culture, or the like and thus two or more samples may be injected together thereinto and mixed therein. Third, the single or mixed cells may be formed and cultured in the hemisphere of the micro-hemisphere array plate and thus various cells may be formed into a three-dimensionally spherical shape under conditions similar to an environment in the human body.

### MODE

Hereinafter, the present invention will be described in detail with reference to exemplary examples in such a manner that it can be easily carried out by one of ordinary skill in the art to which the present invention pertains. However, the present invention may be embodied in many different forms and should not be construed as being limited to examples set forth herein.

### EXAMPLES

### Example 1: Manufacture of Micro-Hemisphere Array Plate and Culture of Cell Aggregate

<Manufacture of Micro-Hemisphere Array Plate>

A photosensitive photoresist was used to form a micro-hemisphere pattern having a size of 500 μm. The photosensitive photoresist used was a negative photoresist, but both negative and positive photoresists may be used as the photosensitive photoresist.

The height of a micro-hemisphere may be adjusted by spin-coating the photosensitive photoresist in a region of 300 μm, and the coated photoresist may be formed into a micro-hemisphere by overcuring at a temperature of 150° C.

A primary seed metal layer was deposited on a micro-hemisphere array pattern formed on a silicon substrate using thin film deposition equipment. In this regard, titanium was used as a seed metal, and the primary seed metal layer was formed to a height of 300 μm. A secondary metal layer included nickel and was formed to a height of 1,500 μm. An E-beam evaporator and a D.C magnetic sputter were respectively used in primary and secondary metal thin film deposition processes.

A nickel layer was formed, on the secondary metal thin film layer, to a large height by electroplating. In this regard, the height of the nickel layer was 0.8 mm and, after electroplating was completed, a rear surface thereof was ground by chemical mechanical planarization (CMP) to obtain uniform flattening.

The grinding-completed nickel layer as well as the secondary metal thin film layer were separated and used as a mold core, and the mold core was mounted in a mold to perform injection molding and then the molding process was performed.

The injection molding process was performed using polystyrene (PS) as a plastic material.

Degrees of hydrophilicity and hydrophobicity of a surface of the completed micro-hemisphere plate were adjusted by oxygen plasma treatment and chemical surface treatment. Through such surface modification, formation of air bubbles in a micro-hemisphere array plate and a micro-hemisphere array microfluidic device was minimized and a three-dimensional hemispherical shape of cells was maximized.

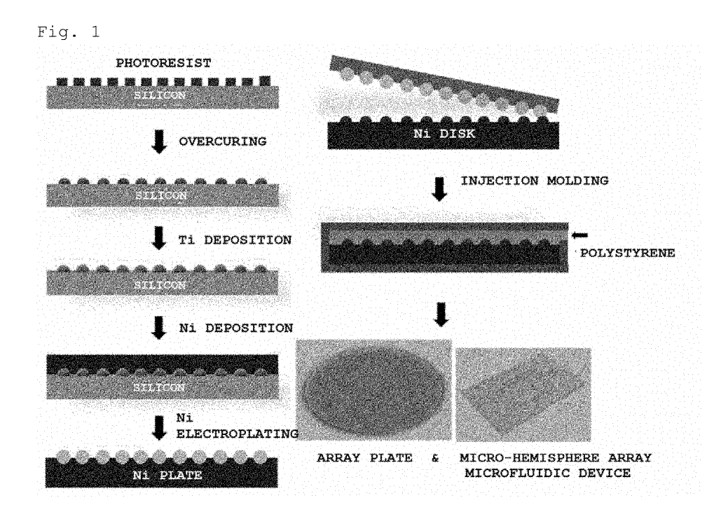

FIG. 1 illustrates a process of manufacturing a micro-hemisphere array plate according to Example 1. FIG. 2A is an image of the micro-hemisphere array plate manufactured by the above-described processes. FIG. 2B is an image of the finally manufactured micro-hemisphere array plate.

<Culture of Cell Aggregate Using Micro-Hemisphere Array Plate>

1) Separation of hADSCs

hADSCs were isolated from adipose tissue removed from a patient having undergone plastic surgery or liposuction. To isolate hADSCs, first, blood fraction was isolated from the isolated adipose tissue. Cells were repeatedly washed with a clean PBS solution until the blood fraction became transparent. Subsequently, Type 1 collagenase was dissolved in PBS at 0.2% and the resulting solution was mixed with the washed cells, thereby breaking intracellular binding and isolating individual cells of a tissue sample. The prepared collagenase solution and the washed adipose tissue were mixed together and incubated while being shaken for one hour. The emulsified tissue was collected and subjected to centrifugation at 600 g for 10 min, and then only pellets were collected and filtered using a 100 μm strainer. The filtered cells were placed in a medium and washed several times and then cultured in a T-75 flask, and, when reaching passage 3-4, the cultured cells were isolated to perform three-dimensional culturing.

2) Culture of hADSCs

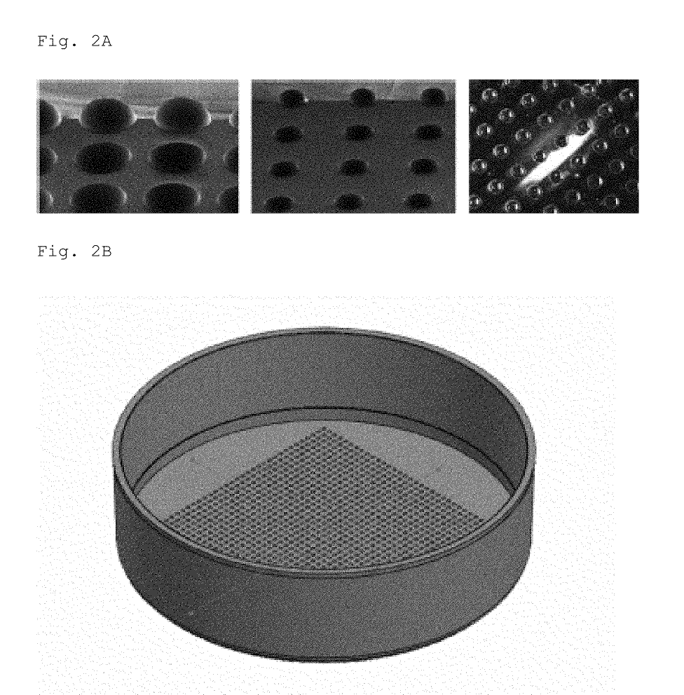

For three-dimensional culturing, hADSCs were placed in a medium, and the cell solution was seeded on the micro-hemisphere array plate. After the cells precipitated in the hemisphere, cells remaining on the surface thereof were removed by washing with a medium, and a new medium was replaced, followed by three-dimensional culturing in an incubator. FIG. 3 illustrates such a culturing process.

3) Three-Dimensional Co-Culturing

FIG. 4 illustrates formation of a cell aggregate by co-culturing a plurality of cells. That is, two types of cells were mixed at a desired ratio and then cultured in a hemisphere as in the above-described processes. 1 day after culturing, the two types of cells were closely linked and combined into a single spherical shape and, accordingly, a completely directly bound three-dimensional co-culturing model was made.

### Example 2: Culturing of Cell Aggregate Using Microfluidic Device Including Micro-Hemisphere Array Plate

<Development of Microfluidic Device Including Micro-Hemisphere Array Plate>

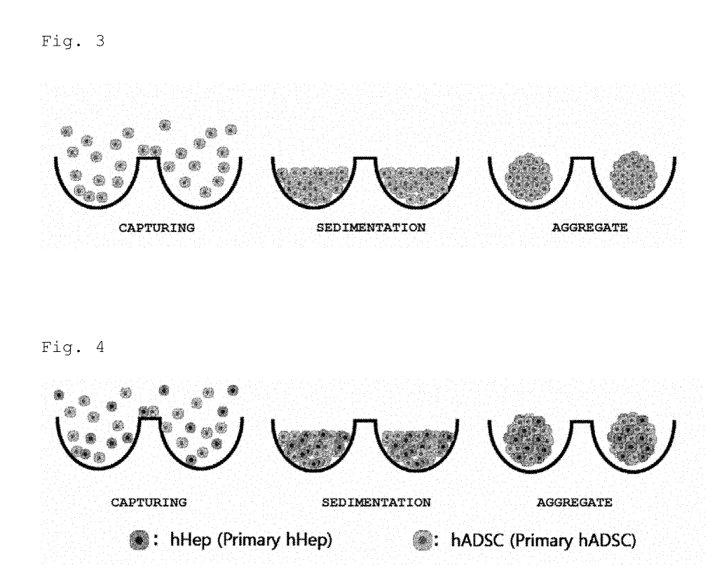

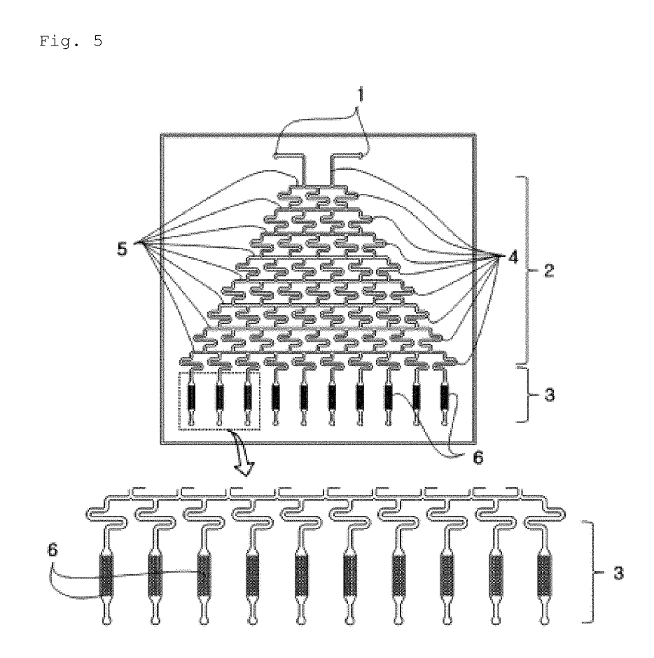

A microfluidic device including the micro-hemisphere array plate manufactured using the method of Example 1 was manufactured. The microfluidic device may include a sample inlet, a sample mixing part, and a cell aggregate formation part including the micro-hemisphere array plate. FIG. 5 is a cross-sectional view of the microfluidic device. FIG. 6 is an image of the manufactured microfluidic device including the micro-hemisphere array plate.

1) Isolation of Human-Derived Hepatocytes

Human hepatocytes were isolated from hepatic tissue removed from a patient having undergone hepatic resection using a conventional collagenase-two-step method. Briefly, first, blood and the like were removed from the isolated liver tissue by EGTA perfusion, and then the resulting liver tissue was perfused with a type 2 collagenase solution so that the collagenase permeated every corner of the tissue, thereby emulsifying the liver tissue. Thereafter, hepatocytes were isolated from the tissue through two washing processes, and the isolated hepatocytes were directly used immediately after isolation.

2) Cell Culturing in Micro-Hemisphere Array Plate Microfluidic Device.

Hepatocytes were mixed with a medium and primary cells and the medium were allowed to slowly pass through a chip at a flow rate of 1 μl/min in a microfluidic device so that the cells precipitated in a hemisphere of a micro-hemisphere array plate, and cells remaining around a micro-hemisphere array were effectively removed using this flow rate. This process was repeated numerous times to culture human-derived primary cells in the microfluidic device including the micro-hemisphere array plate so that the cells were grown into a three-dimensional spherical shape in the microfluidic device.

3) Three-Dimensional Co-Culturing

By using the same method as that described above, cells were loaded such that two or more types of cells were mixed at a desired ratio and added together with a medium. As such, three-dimensional co-culturing may be performed in a microfluidic device having a flow rate.

Meanwhile, such a microfluidic device including a micro-hemisphere array plate has three main functions.

First, the microfluidic device has a concentration gradient function in which, by adding a culture for culturing cells and a sample capable of inducing cell differentiation and maximizing characteristic variations or functions of cells, the culture and the sample can flow into each chamber according to concentration.

Second, the microfluidic device has a micromixer function capable of uniformly mixing two or more solutions because a culture and a functional sample are both added.

Third, the microfluidic device can form various cells into a three-dimensional cell spheroid by integrating cells into hemispheres of a micro-hemisphere array plate.

Thus, optimum microfluidic environments for three-dimensional cell culture may be provided by freely varying a fluid in a region of 10 nl/min to 10 μl/min in a microchannel.

### COMPARATIVE EXAMPLES

### Comparative Example 1

Cells were cultured using the same method as that used in Example 1, except that cells were cultured in a hemispherical micro-well manufactured using an existing method.

### Comparative Example 2

A cell aggregate was two-dimensionally cultured through an existing method without injection and transfer of a sample.

### EXPERIMENTAL EXAMPLES

### Experimental Example 1: Observation of Cell Aggregate Cultured in Micro-Hemisphere Array Plate

A cell aggregate cultured in the micro-hemisphere array plate of Example 1 and a cell aggregate cultured according to Comparative Example 1 were observed. The observation results can be confirmed in FIGS. 7A-7C and 8A-8B.

In FIG. 7A, it can be confirmed that on 1 day after hADSCs were added to the micro-hemisphere array plate of Example 1, a spherically agglomerated cell spheroid was satisfactorily formed. In addition, in FIG. 7B, it can be confirmed that the formed cell spheroid was grown for 9 days and then the viability thereof was evaluated by Live/Dead assay and, as a result, most cells were alive and healthy. In addition, in FIG. 7C, it can be confirmed that the formed cell spheroid was collected on the 9^{th} day and a fine structure thereof was examined through an SEM image and, as a result, the cell spheroid exhibited microvilli, which is a characteristic of hADSCs, and agglomerated cells were formed into a single, completely spherical shape. Such a micro-hemisphere array plate may be manufactured in a desired number and a large area, and thus may be readily mass-produced. In addition, a manufacturing method thereof is also very simple and thus is very suitable for use in rapid and easy mass-production of hADSC spheroids.

Meanwhile, in FIG. 8A, it was confirmed that, when hADSCs were grown as Comparative Example 1, the hADSCs became adherent cells, which are adhered to a surface, over 10 days after formation of a cell sphere, and thus the formed cell sphere escaped from micro-holes, resulting in adhesion to the floor. However, as can be confirmed in FIG. 8B, in the case of Example 1, it is shown that height adjustment is possible and thus cell spheres remain in the original place over time in a chip manufactured to a large depth and long-term storage thereof in a stable state is possible. This indicates that cells not having aggregation characteristics may also agglomerate satisfactorily and may be grown into a spherical state for a long period of time through depth adjustment.

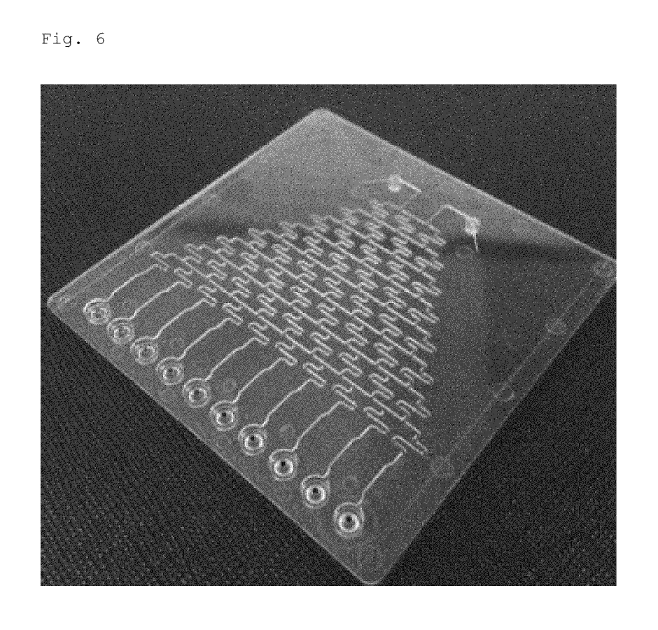

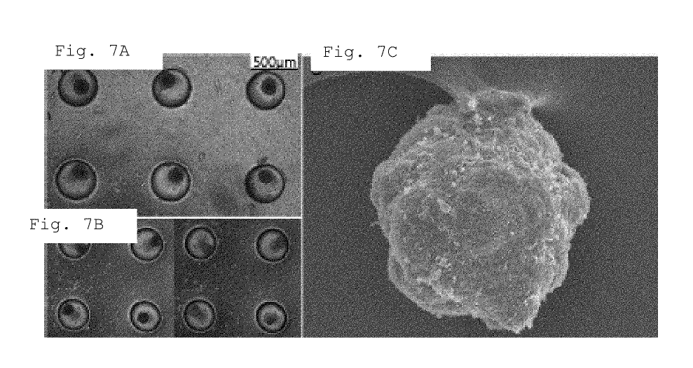

FIGS. 9A-9E illustrate culturing results of the hADSCs under conditions: 2D as in Comparative Example 2 (see FIGS. 9A and 9B), 3D as in Example 1 (see FIGS. 9C and 9D), optical (see FIGS. 9A and 9C), GFP (see FIGS. 9B and 9D), and SEM (see FIG. 9E).

### Experimental Example 2: Observation of Cell Aggregate Cultured in Microfluidic Device Including Micro-Hemisphere Array Plate

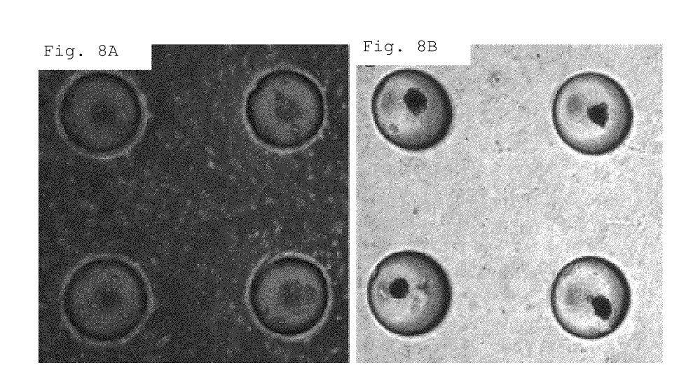

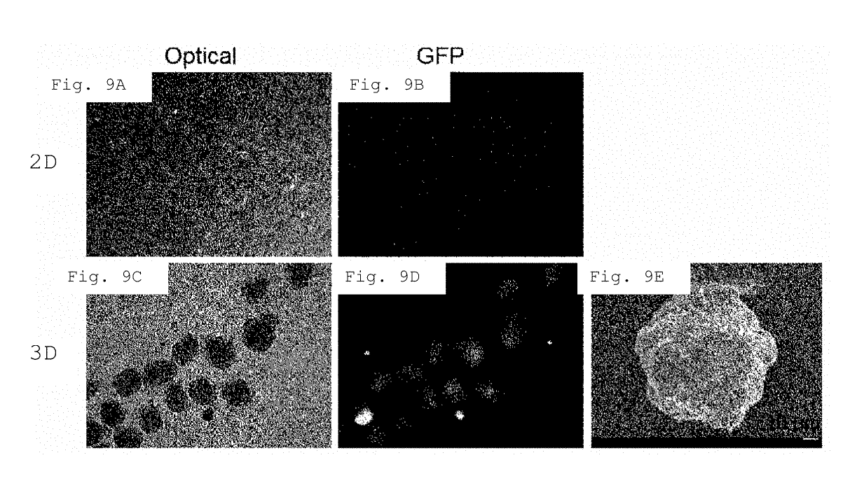

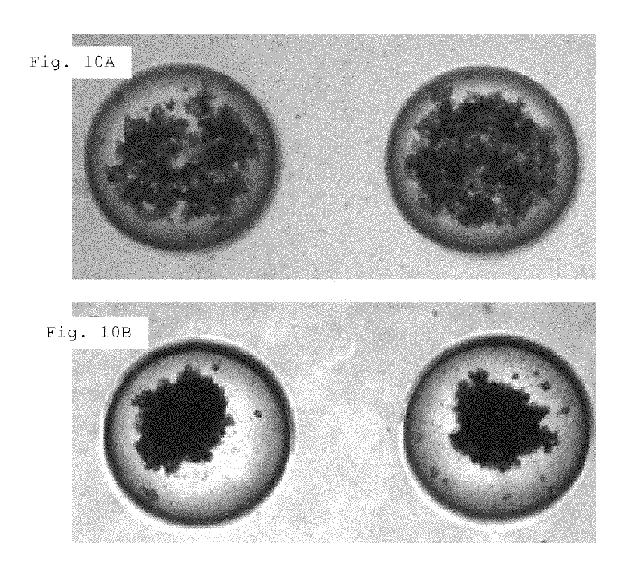

Human liver cells and a sample were injected through the microfluidic device of Example 2 and a cell aggregate cultured in the presence of fluid flow (see FIG. 10B) and a cell aggregate cultured in a micro-hemisphere array plate in the absence of fluid flow (see FIG. 10A) were observed.

In the case of the human liver, cells obtained from a partial hepatectomy are in poor condition, and thus do not readily form an aggregation regardless of growth method employed.

As can be confirmed in FIG. 10A, when a human liver cell aggregate was formed in the absence of a flow rate similar to the human body, aggregation hardly occurred.

In contrast, as can be confirmed in FIG. 10B, in the presence of fluid flow through the microfluidic device of Example 2, even cells in poor condition may form an aggregate. Thus, in the microfluidic device including a micro-hemisphere array plate, human primary cells which are difficult to obtain in good condition may be effectively cultured three-dimensionally, and general cells as well as cells in poor condition may be beneficially affected by applying continuous flow and shear stress. This enables formation of an environment similar to that in the human body, thereby maximizing normal functions of cells and continuously providing fresh medium due to continuous flow of a fluid. In addition, the three characteristics of the microfluidic device may be utilized in continuous drug screening by flowing a drug for inducing cell differentiation and analyzing functions of cells.

In addition, FIGS. 11A-11B illustrate images showing measurement results of an experiment for identifying human hepatocytes exhibiting activity by staining after such cell culturing was maintained for 3 days. As shown in FIG. 11A, in Comparative Example 2, when human primary hepatocytes in poor condition were cultured in the absence of fluid flow, over half of the cells were dead as can be confirmed through Live/Dead assay, and the cells were not agglomerated well and not stained well. In contrast, in Example 2 (see FIG. 11B) with fluid flow, it can be confirmed that the viability of the cells was significantly different from the results of Comparative Example 2 without fluid flow. Considering that the viability of human liver cells initially isolated was about 40% to about 60%, the viability was rather increased over time in Example 2 without fluid flow, from which it can be confirmed that live cells remained and were strongly agglomerated to thereby increase overall viability. This demonstrated that, when poorly conditioned cells were cultured in Example 2 with fluid flow, their condition was improved, and this indicates the possibility of using, in experiments, human primary cells, which are in poor condition but the most readily available cell source.

FIGS. 12A-12F illustrate images showing results of culturing human liver cells under conditions: 2D as in Comparative Example 2 (see FIGS. 12A and 12B), 3D as in Example 2 (see FIGS. 12C and 12D), optical (see FIGS. 12A and 12C), ALB (see FIGS. 12B and 12D), Live/Dead (see FIG. 12E), and SEM (see FIG. 12F).

### Experimental Example 3: Three-Dimensional Co-Culture Through Direct Binding of a Plurality of Human Cells

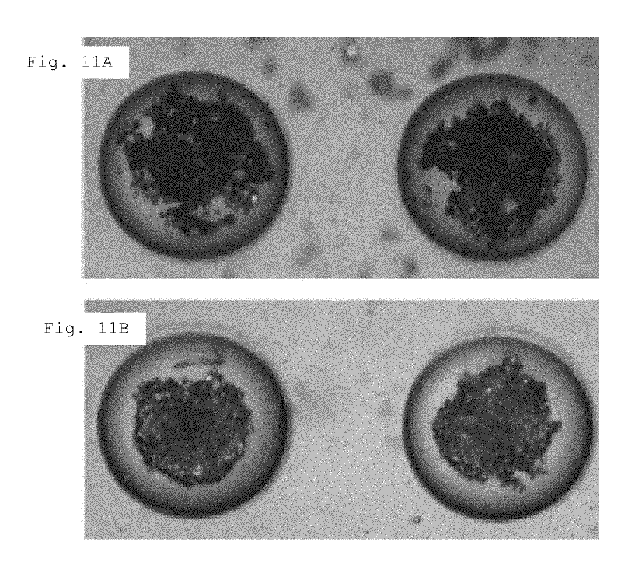

An experiment was conducted to identify, when human liver cells and hADSCs were mixed and grown in the micro-hemisphere array plate of Example 1, the possibility of three-dimensional co-culture by direct binding of these cells. The results are illustrated in FIGS. 13A-13D. FIG. 13A illustrates that two types of cells are agglomerated to form an aggregate. FIG. 13B illustrates that a single sphere consisting of two types of cells has very high viability, as expressed in green, and thus is cultured in a very healthy hemisphere. FIG. 13C is an SEM image taken on the third day after culturing, from which it can be confirmed that the two types of cells were agglomerated into a completely single form without any boundary or separation. FIG. 13D is an image taken on the ninth day after culturing, from which it can be confirmed that the two types of cells were more strongly agglomerated into a single form than on the third day and a boundary therebetween was completely disappeared. This indicates that two types of cells are directly bound to each other to form a complete single unit, which may be a three-dimensional co-culture model by new direct binding.

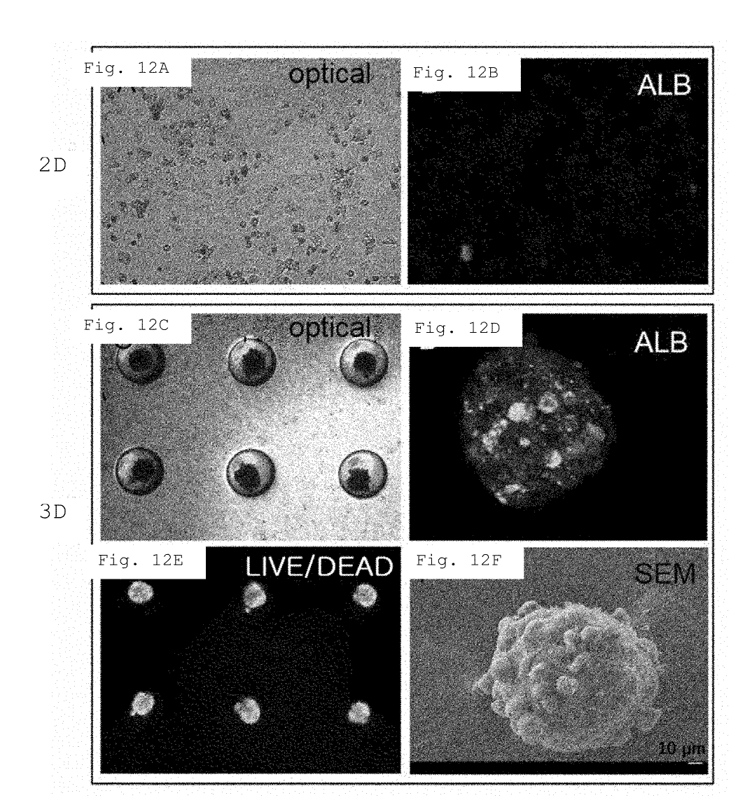

FIGS. 14A-14E illustrate results of performing a function test using a three-dimensional co-culture model by such direct binding. In FIGS. 14A and 14B, it can be confirmed that, as in general hepatocytes, the model exhibited activated secretion of albumin (see FIG. 14A) and urea (see FIG. 14B), which indicates that it functions well even in a mixed state of agglomerated cells. In FIGS. 14C and 14D, a high level was shown even in Cytochrome P450 reductase staining, as expressed in red, and a continuously high level was shown in the CYP3A4 activity quantification graph illustrated in FIG. 14E, from which it can be confirmed that the cell sphere performed well, even with regard to functions related to liver-specific metabolism. Through showing that a new unit of the agglomerated cells functionally performs its role, conditions similar to various intracellular bindings in actual organs were realized, which shows that such a unit may be usefully used in vitro and in vivo.

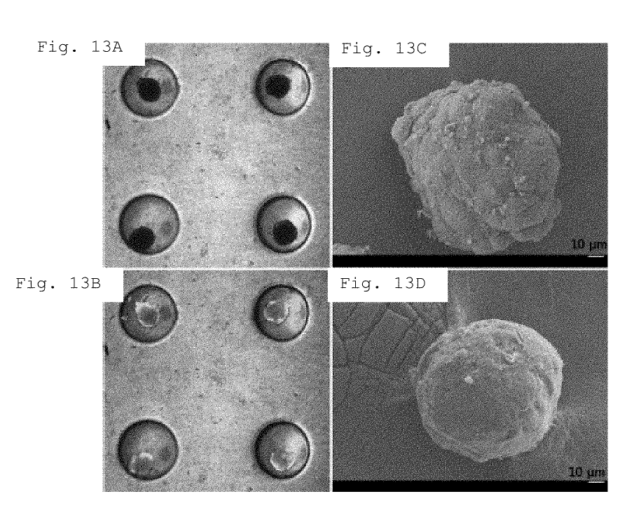

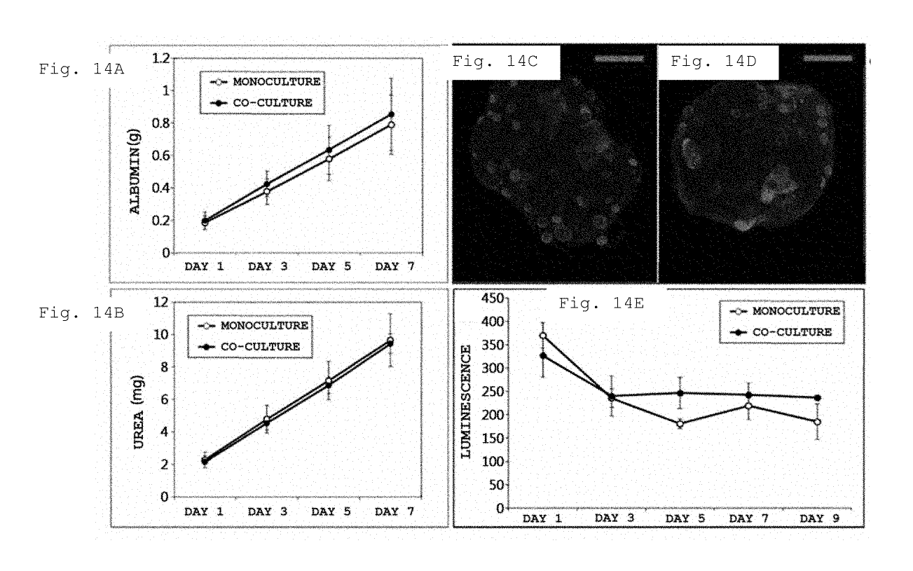

In addition, as can be confirmed from FIGS. 15A-15F, when the inside of a co-cultured cell sphere was photographed by a TEM, the cell sphere was confirmed to exhibit various characteristics of activated cells. Many mitochondria, healthy vasculature, tight junctions specific to hepatocytes, bile canaliculi, and the like are observed, and glycogen and collagen, which form the ECM, can also be observed. Peroxisome and rough ER were also identified and endocytosis was observed, from which it can be confirmed that the cells were morphologically and functionally very healthy.

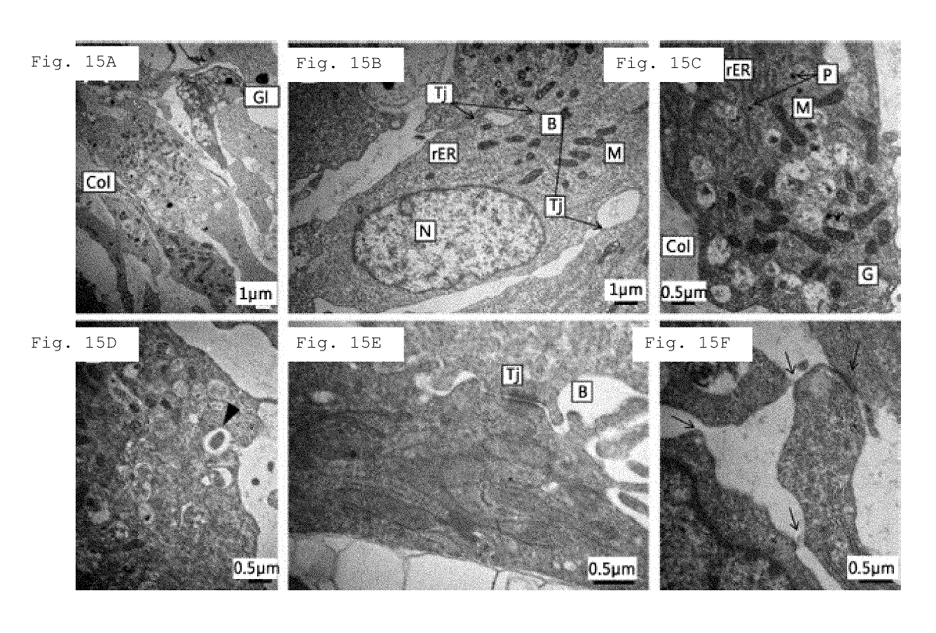

FIGS. 16A-16B illustrate images showing the viability of cells through performing Live/Dead assay on a poorly conditioned hepatocyte sphere (see FIG. 16A) and a three-dimensionally co-cultured cell sphere by direct binding (see FIG. 16B). As can be seen in FIGS. 16A-16B, most cells were alive and the number of dead cells was very small, from which it can be confirmed that cells were not damaged in a process of taking the cells out of the micro-hemisphere array plate of Example 1, and this indicates that, in addition to three-dimensional culturing of cells in hemispheres, the cells may be used in other desired places by using different methods after removal therefrom.

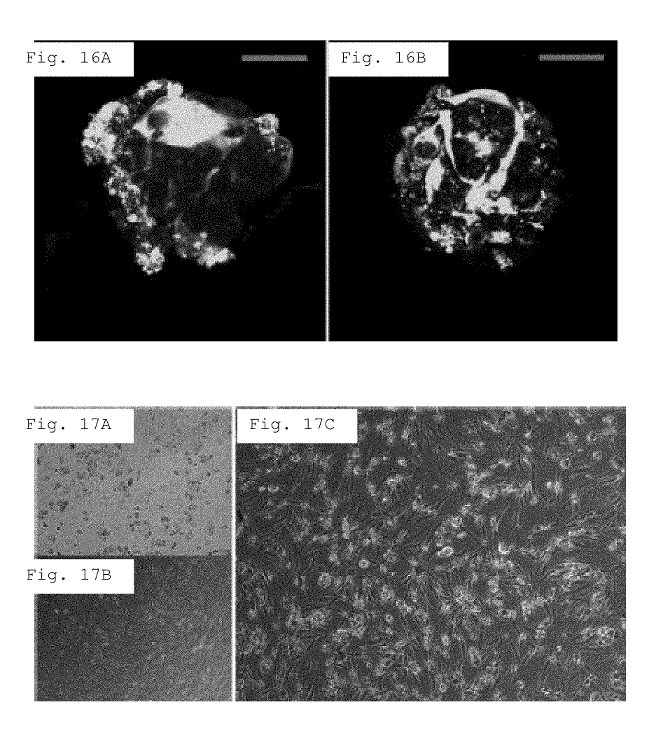

FIGS. 17A-17C illustrate images of human primary hepatocytes (see FIG. 17A), hADSCs (see FIG. 17B), and the two types of cells co-cultured on 2D (see FIG. 17C). From the results, it can be confirmed that, although directly bound to each other, the two types of cells did not exhibit an effect of forming a single unit and were co-cultured to a degree to which the two types of cells were adhered to each other in one space. The activity of the cells was identified such that an albumin secretion site was stained using the two-dimensionally co-cultured model (see FIGS. 18A-18B), from which it can be confirmed that the model exhibited little activity, as expressed in red.

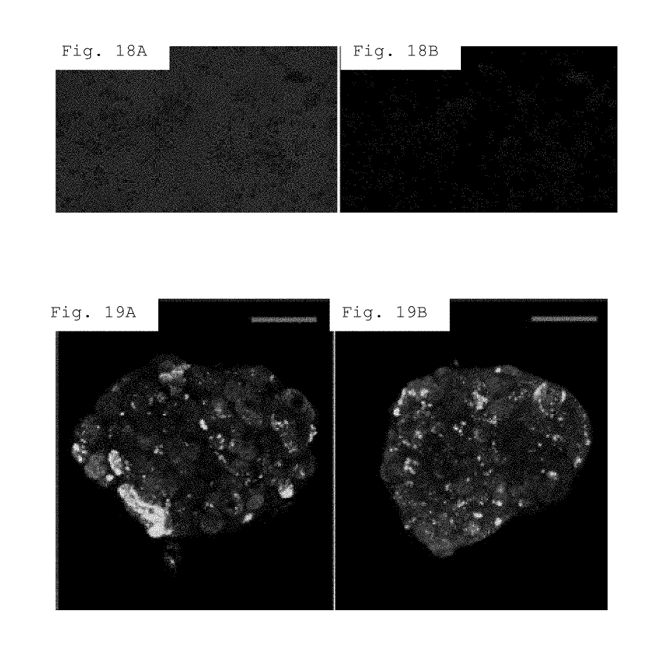

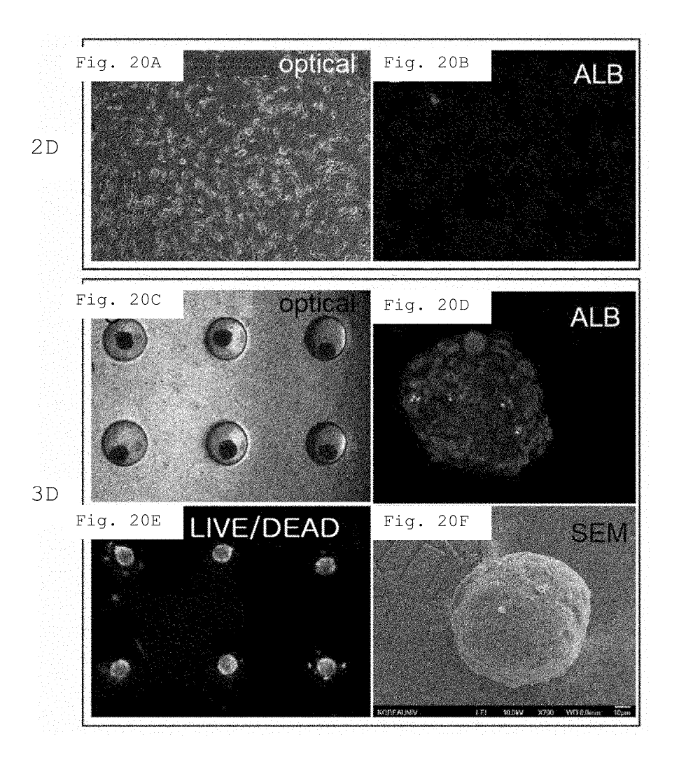

In contrast, it can be confirmed that, when the cells were co-cultured in the micro-hemisphere array plate manufactured according to Example 1 (see FIGS. 19A-19B), albumin secretion, expressed in green, was very active and the activity of the cells was much better. Meanwhile, FIGS. 20A-20F illustrate images comprehensively showing the case of a 2D environment (see FIGS. 20A and 20B) and the case of a 3D environment as in Examples of the present invention.

Although exemplary embodiments of the present invention have been described, the present invention should not be construed as limited to the embodiments set forth therein. The present invention may be embodied in many different forms within the sprit and scope of the present invention and it is obvious that these changes are also within the scope of the appended claims.

### DESCRIPTION OF REFERENCE NUMERALS

1: Sample inlet
2: Sample mixing part
3: Cell aggregate formation part
4: Channel
5: Flow channel

What is claimed is:

1. A method of manufacturing a micro-hemisphere array plate, the method comprising:
1) attaching a photosensitive photoresist to a silicon substrate;
2) adjusting a height of the photosensitive photoresist by spin coating;
3) hemispherically etching the photoresist by overcuring;
4) depositing a primary metal layer on an etched surface of the photoresist after step 3);
5) depositing a secondary metal layer on the primary metal layer after the depositing;
6) forming a mold core layer on the secondary metal layer;
7) planarizing an upper surface of the mold core layer after step 6);
8) separating the mold core layer after step 7);
9) injection-molding a micro-hemisphere array plate by using the separated mold core layer as a mold; and
10) imparting hydrophilicity or hydrophobicity to a surface of the micro-hemisphere array plate.

2. The method of claim 1, wherein the photosensitive photoresist has a length of 100 μm to 1,000 μm.

3. The method of claim 1, wherein the primary metal layer comprises at least one selected from the group consisting of Cr, Ti, Au, Ni, Cu, Al, and Fe.

4. The method of claim 1, wherein the secondary metal layer comprises at least one selected from the group consisting of Au, Ag, Pt, Ni, and Cu.

5. The method of claim 1, wherein the mold core layer comprises at least one selected from the group consisting of nickel, titanium, and aluminum.

6. The method of claim 1, wherein the injection-molding is performed using at least one selected from the group consisting of polycarbonate (PC), polymethylmethacrylate (PMMA), polystyrene (PS), and cyclic olefin copolymer (COC).

7. The method of claim 1, wherein a hemisphere of the micro-hemisphere array plate has a diameter of 100 μm to 1,000 μm.

8. A cell aggregate cultured using the micro-hemisphere array plate manufactured by the method according to claim 1.

9. A method of culturing a cell aggregate by using the micro-hemisphere array plate manufactured by the method according to claim 1.

10. A microfluidic device comprising a micro-hemisphere array plate, the microfluidic device comprising:
a sample inlet through which a sample comprising a single cell or a plurality of cells and a cell culture is injected via a single or plurality of channels;
a sample mixing part connected to the sample inlet, allowing the sample to be mixed therein while moving, wherein the moving is performed via a single or plurality of channels, the single or plurality of channels having a zigzag form, the single or plurality of channels being repetitively disposed in a pyramid form through a plurality of steps, and the steps being configured such that a lower step further comprises one or more channels than in an upper step, and comprising a flow channel connecting the steps to one another; and
a cell aggregate formation part connected to the sample mixing part, connected to channels constituting the lowermost step among the steps, and comprising a plurality of micro-hemisphere array plates in which the single or mixed cells in the mixed sample are three-dimensionally cultured to form a cell aggregate.

11. The microfluidic device of claim 10, wherein the micro-hemisphere array plate is a micro-hemisphere array plate manufactured by the method according to claim 1.

12. The microfluidic device of claim 10, wherein the sample is injected via the sample inlet at a flow rate of 10 nl/min to 10 μl/min.

13. The microfluidic device of claim 10, wherein the channel has a diameter of 500 μm to 2.0 mm.

14. The microfluidic device of claim 10, wherein a hemisphere of the micro-hemisphere array plate has a diameter of 100 μm to 1,000 μm.

15. A cell aggregate cultured using the microfluidic device according to claim 10.

16. A method of culturing a cell aggregate by using the microfluidic device according to claim 10.
